(19) United States
(12) Patent Application Publication
(10) Pub. No.: US 2018/0028160 A1
(43) Pub. Date: Feb. 1, 2018

(54) ULTRASONIC DIAGNOSTIC APPARATUS

(71) Applicant: SAMSUNG MEDISON CO., LTD., Gangwon-do (KR)

(72) Inventors: Joo Yeon CHO, Chuncheon-si (KR); Kwang-Jung LEE, Wonju-si (KR)

(21) Appl. No.: 15/372,265

(22) Filed: Dec. 7, 2016

(30) Foreign Application Priority Data
Jul. 29, 2016 (KR) 10-2016-0096680

(51) Int. Cl. A61B 8/00

(52) U.S. Cl. CPC A61B 8/56; A61B 8/461; A61B 8/467; A61B 2562/0271

(57) ABSTRACT

An ultrasonic diagnostic apparatus may include a main body; and at least one input/output unit coupled with the main body, and configured to receive information from a user or to output information received from the main body; and a connection unit configured to connect the main body to the input/output unit and the connection unit comprises a support member configured to support the input/output unit, and at least one connection member configured to adjust a magnitude of a supporting force generated by the support member. The connection member may generate a force in a direction in which the supporting force generated by the support member is cancelled out to thus adjust the magnitude of the supporting force.

### CROSS-REFERENCE TO RELATED APPLICATION

This application claims the benefit of Korean Patent Application No. 10-2016-0096680, filed on Jul. 29, 2016 in the Korean Intellectual Property Office, the disclosure of which is incorporated herein by reference.

### BACKGROUND

### 1. Field

Embodiments of the present disclosure relate to an ultrasonic diagnostic apparatus, and more particularly, to a technique for enabling a user to easily move an input/output unit including a control panel and a display to adjust the height of the input/output unit, in consideration of the user's physical condition and a surrounding environment.

### 2. Description of the Related Art

An ultrasonic diagnostic apparatus irradiates ultrasound signals to a target area inside an object, and receives ultrasound signals (that is, ultrasound echo signals) reflected from the object so as to non-invasively acquire section images about soft tissue of the object or images about blood vessels of the object based on the ultrasound echo signals.

The ultrasonic diagnostic apparatus has an advantage that it is a compact, low-priced apparatus compared to other medical imaging apparatuses, such an X-ray diagnostic apparatus, an X-ray Computerized Tomography (CT) scanner, a Magnetic Resonance Imaging (MRI) apparatus, and a nuclear medicine diagnosis apparatus.

Also, the ultrasonic diagnostic apparatus can display images about the inside of an object in real time, and has high safety since there is no risk for patients to be exposed to radiation. For the advantages, the ultrasonic diagnostic apparatus is widely used to diagnose the heart, abdomen, urinary organs, uterus, etc.

The ultrasonic diagnostic apparatus includes a probe to transmit ultrasound signals to a main body and an object and to receive ultrasound signals reflected from the object, a display disposed above the main body and configured to display images of the results of diagnosis obtained from the received ultrasound signals, and a control panel disposed in front of the display and configured to enable a user to manipulate the ultrasonic diagnostic apparatus. The control panel and the display correspond to an input/output unit for inputting or outputting information.

The ultrasonic diagnostic apparatus can include a height adjusting apparatus for adjusting the height of the input/output unit according to a user's physical condition or a surrounding environment, since many users using the ultrasonic diagnostic apparatus have different physical conditions.

The height adjusting apparatus includes an automatic type of adjusting the height of the input/output unit using power generated by a driving member, and a manual type of adjusting the height of the input/output unit according to a user's force.

In the case of the manual type, a user applies a force to adjust the height of the input/output unit to his/her desired height. However, since typical manual types do not consider a user's physical condition, the user experiences inconvenience in moving the input/output unit.

That is, if the user is a woman or weak, the user has difficulties in moving the input/output unit since the supporting force of a connection member supporting the input/output unit is strong. In contrast, if the user is a man or strong, the user can move the input/output unit too easily, which may result in failure in adjusting the height of the input/output unit to his/her desired height.

Also, if the temperature of a surrounding environment, for example, an environment where the ultrasonic diagnostic apparatus is located is too high or low, the supporting force of a gas spring supporting the input/output unit changes. However, the typical types do not reflect such a change in supporting force of the gas spring properly so that a user can have difficulties in moving the input/output unit.

### SUMMARY

Therefore, it is an aspect of the present disclosure to provide an ultrasonic diagnostic apparatus which is capable of changing the magnitude of a supporting force supporting an input/output unit according to a user's physical condition or a surrounding environment so that the user can easily move the input/output unit.

Additional aspects of the disclosure will be set forth in part in the description which follows and, in part, will be obvious from the description, or may be learned by practice of the disclosure.

In accordance with one aspect of the present disclosure, an ultrasonic diagnostic includes a main body; at least one input/output unit coupled with the main body, and configured to receive information from a user or to output information received from the main body; and a connection unit configured to connect the main body to the input/output unit, and the connection unit comprises a support member configured to support the input/output unit, and at least one connection member configured to adjust a magnitude of a supporting force generated by the support member.

The connection member may generate a force in a direction in which the supporting force generated by the support member is cancelled out to thus adjust the magnitude of the supporting force.

The support member may include a gas spring.

The connection member may include an elastic member having elasticity.

The ultrasonic diagnostic apparatus may further include an adjusting unit coupled with one end of the connection member, and configured to adjust a length of the connection member.

The adjusting unit may include an adjusting screw configured to rotate to adjust the length of the connection member.

The adjusting unit further may include a handle coupled with the adjusting screw, and rotation of the adjusting screw is adjusted by the handle.

The adjusting unit may include a lever handle configured to adjust the length of the connection member based on the principle of the lever.

The ultrasonic diagnostic apparatus may include a fixing unit configured to fix the adjusting unit or to prevent reverse rotation of the adjusting unit.

The connection member may include a single or a plurality of magnetic members having magnetism.

The magnitude of the supporting force may be adjusted according to an interval between the plurality of magnetic members.

The connection member may further include a coil wound around the single or the plurality of magnetic members, and the magnitude of the supporting force may be adjusted according to a magnitude of current flowing through the coil.

The ultrasonic diagnostic apparatus may further include an input unit configured to receive information about an interval between the plurality of magnetic members or information about a magnitude of the current flowing through the coil, from the user.

The ultrasonic diagnostic apparatus may further include a controller configured to control the interval between the plurality of magnetic member or the magnitude of the current flowing through the coil, according to the received information.

The ultrasonic diagnostic apparatus may further include an input unit configured to receive information about the length of the connection member from the user.

The input unit may include one of a button, a switch, a knob, and a jog-dial.

The ultrasonic diagnostic apparatus may further include a motor configured to adjust the length of the connection member, and a controller configured to control operation of the motor and the controller adjusts the length of the connection member using the motor based on the received information.

The ultrasonic diagnostic apparatus may further include a sensor configured to measure outside temperature, and a controller configured to adjust the length of the connection member based on the outside temperature measured by the sensor.

The ultrasonic diagnostic apparatus may further include a storage unit configured to store information about the connection member set by the user, and a display configured to display a magnitude of a force generated by the connection member.

The input/output unit may include one of a control panel and a display.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 shows an external configuration of an ultrasonic diagnostic apparatus according to an embodiment of the present disclosure.

FIG. 2 is a side view of a typical ultrasonic diagnostic apparatus

FIG. 3 is a view for describing various forces that are applied to a control panel of the typical ultrasonic diagnostic apparatus.

FIG. 4 is a graph showing the magnitude of a force generated by a gas spring according to a change in temperature.

FIG. 5 is an exploded perspective view showing a part of a connection unit of an ultrasonic diagnostic apparatus according to an embodiment of the present disclosure.

FIG. 6 is an exploded perspective view showing the part of the connection unit of the ultrasonic diagnostic apparatus according to an embodiment of the present disclosure.

FIG. 7 is a view for describing a relationship between an elastic force and an external force.

FIG. 8 shows the direction and magnitude of a force applied to the connection unit according to an embodiment of the present disclosure.

FIG. 9 shows the adjusting unit for adjusting the length of the connection member according to an embodiment of the present disclosure.

FIG. 10 shows the various device for adjusting unit according to an embodiment of the present disclosure.

FIG. 11 shows the adjusting unit including a lever handle according to an embodiment of the present disclosure.

FIG. 12 is a view for describing a case of adjusting the length of a connection member using a motor, according to another embodiment of the present disclosure.

FIG. 13 shows a connection member including one or more magnetic members, according to another embodiment of the present disclosure.

FIG. 14 is a view for describing a force generated by the connection member including the magnetic members according to another embodiment of the present disclosure.

FIG. 15 is a block diagram showing components constituting a part of an ultrasonic diagnostic apparatus according to another embodiment of the present disclosure.

FIG. 16 shows an input unit and display of according to another embodiment of the present disclosure.

FIG. 17 shows an input unit and storage unit according to another embodiment of the present disclosure.

### DETAILED DESCRIPTION

Configurations illustrated in the embodiments and the drawings described in the present specification are only the preferred embodiments of the present disclosure, and thus it is to be understood that various modified examples, which may replace the embodiments and the drawings described in the present specification, are possible when filing the present application.

The terms used in the present specification are used to describe the embodiments of the present disclosure. Accordingly, it should be apparent to those skilled in the art that the following description of exemplary embodiments of the present invention is provided for illustration purpose only and not for the purpose of limiting the invention as defined by the appended claims and their equivalents. It is to be understood that the singular forms “a,” “an,” and “the” include plural referents unless the context clearly dictates otherwise.

In this specification, it will be understood that when the terms “includes,” “comprises,” “including,” and/or “comprising,” when used in this specification, specify the presence of stated features, figures, steps, components, or combination thereof, but do not preclude the presence or addition of one or more other features, figures, steps, components, members, or combinations thereof.

It will be understood that, although the terms first, second, etc. may be used herein to describe various components, these components should not be limited by these terms. These terms are only used to distinguish one component from another. For example, a first component could be termed a second component, and, similarly, a second component could be termed a first component, without departing from the scope of the present disclosure. As used herein, the term “and/or” includes any and all combinations of one or more of associated listed items.

Hereinafter, the configuration of the present disclosure will be described in detail with reference to the accompanying drawings so that one of ordinary skill in the art can easily embody the present disclosure.

In the drawings, the thicknesses of several layers and pixel areas are enlarged in order to definitely show the layers and the pixel areas. It will also be understood that when an element such as a layer, film, pixel region or plate is referred to as being “on” or “over” another element, it can be directly on the other element or intervening elements may also be present.

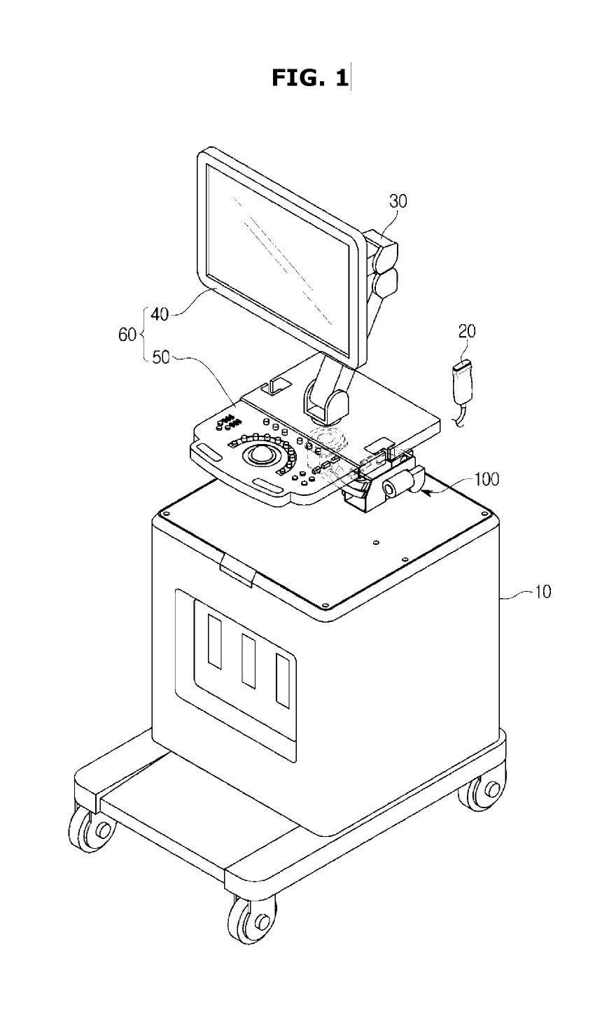

FIG. 1 shows an external configuration of an ultrasonic diagnostic apparatus according to an embodiment of the present disclosure.

As shown in FIG. 1, an ultrasonic diagnostic apparatus 1 according to an embodiment of the present disclosure may include a main body 10, an ultrasound probe 20 configured to transmit an ultrasound signal to an object that is to be diagnosed, and to receive an ultrasound signal reflected from the object, a display 40 disposed above the main body 10, and configured to output results obtained from the received ultrasound signal as an image, a control panel 50 configured to enable a user to input various commands for manipulating the ultrasonic diagnostic apparatus 1, and a connection unit 100 connecting the control panel 50 or the display 40 to the main body 10.

Also, the ultrasonic diagnostic apparatus 1 may include a moving element 30 positioned between the display 40 and the main body 10, and configured to move the display 40. The moving element 30 may rotate or move the display 40.

According to an embodiment of the present disclosure, the ultrasound probe 20 may be connected to the main body 10 through a wired or wireless communication network to receive various signals required for the control of the ultrasound probe 20 or to transfer an analog signal or a digital signal corresponding to an echo ultrasound signal received by the ultrasound probe 20.

Meanwhile, the wireless communication network means a communication network to transmit and receive signals in a wireless fashion, and the main body 10 can perform wireless communication with the ultrasound probe 20 through at least any one of a short-range communication module and a mobile communication module.

The short-range communication module means a module for short-range communication within a predetermined distance. For example, short-range communication technology includes a Wireless Local Area Network (WLAN), Wireless-Fidelity (Wi-Fi), Bluetooth, Zigbee, Wi-Fi Direct (WFD), Ultra WideBand (UWB), Infrared Data Association (IrDA), Bluetooth Low Energy (BLE), and Near Field Communication (NFC), although it is not limited to these.

The mobile communication module may transmit and receive radio signals to and from at least any one of a base station, an external terminal, and a server on a mobile communication network. Herein, the radio signals means signals including various types of data. That is, the main body 10 can transmit and receive various types of data to and from the ultrasound probe 20 via at least one of a base station and a server.

For example, the main body 10 may transmit and receive signals including various types of data to and from the ultrasound probe 20 via a base station using a mobile communication network, such as 3Generation (3G) or 4Generation (4G).

The main body 10 may receive/transmit data from/to a hospital server or other medical apparatuses in a hospital, connected through Picture Archiving and Communication System (PACS). Also, the main body 10 may transmit and receive data according to a Digital Imaging and Communications in Medicine (DICOM) standard, although it is not limited to this.

Also, the main body 10 may transmit and receive data to and from the ultrasound probe 20 through a wired communication network. The wired communication network means a communication network to transmit and receive signals in a wired fashion.

According to an embodiment of the present disclosure, the main body 10 may transmit and receive various types of signals to and from the ultrasound probe 20 through a wired communication network, such as Peripheral Component Interconnect (PCI), PCI-express, a Universal Serial Bus (USB), and the like, although it is not limited to this.

Meanwhile, the main body 10 may include the display 40 and the control panel 50. The control panel 50 or the display 40 may receive various control commands for controlling the ultrasonic diagnostic apparatus 1, as well as setting information about the ultrasound probe 20, from a user.

According to an embodiment, the setting information about the ultrasound probe 20 may include gain information, zoom information, focus information, Time Gain Compensation (TGC) information, depth information, frequency information, power information, frame average information, dynamic range information, etc. However, the setting information about the ultrasound probe 20 is not limited to the above-mentioned information, and may include various information that can be set in order to photograph ultrasound images.

The information may be transferred to the ultrasound probe 20 through the wireless communication network or the wired communication network, and the ultrasound probe 20 may be set according to the received information. Also, the main body 10 may receive various control commands, such as a command for transmitting an ultrasound signal, through the control panel 50 or the display 40, from the user, and transfer the various control commands to the ultrasound probe 20.

The display 40 may be implemented as a device well-known to those skilled in the art, such as a Cathode Ray Tube (CRT) display, a Liquid Crystal Display (LCD), a Light Emitting Diode (LED) display, a Plasma Display Panel (PDP) display, an Organic Light Emitting Diode (OLED) display, etc., although not limited to these.

The display 40 may display an ultrasound image of a target area inside the object. The ultrasound image displayed on the display 40 may be a 2Dimensional (2D) ultrasound image or a 3Dimensional (3D) ultrasound image. That is, the display 40 may display various ultrasound images according to operation modes of the ultrasonic diagnostic apparatus 1.

Also, the display 40 may display information about operation states of the ultrasound probe 20, as well as a menu or guidance for ultrasonic diagnosis.

According to an embodiment, the ultrasound image may include an Amplitude mode (A-mode) image, a Brightness mode (B-mode) image, a motion mode (M-mode) image, a Color mode (C-mode) image, and a Doppler mode (D-mode) image.

The A-mode image, which is described below, means an ultrasound image representing the amplitude of an ultrasound signal corresponding to an echo ultrasound signal, the B-mode image means an ultrasound image representing the amplitude of an ultrasound signal corresponding to an echo ultrasound signal, as brightness, and the M-mode image means an ultrasound image representing motion of an object according to time at a specific location. The D-mode image means an ultrasound image representing a moving object in the form of a waveform using the Doppler effect, and the C-mode image means an ultrasound image representing a moving object in the form of a color spectrum.

Meanwhile, if the display 40 is implemented as a touch screen type, the display 40 may perform the functions of the control panel 50. That is, the main body 10 may receive various commands from the user through at least one of the display 40 and the control panel 50.

The control panel 50 may be implemented as a keyboard, a foot switch, or a foot pedal.

For example, the keyboard may be hardwarily implemented. The keyboard may include at least one of a switch, a key, a joystick, and a trackball. According to another example, the keyboard may be softwarily implemented, like a graphic user interface. In this case, the keyboard may be displayed through the display 40.

The foot switch or the foot pedal may be disposed below the main body 10, and the user may control operations of the ultrasonic diagnostic apparatus 1 using the foot switch or the foot pedal.

Also, the main body 10 may include a voice recognition sensor to receive voice commands from the user, which is not shown in the drawings.

Also, the display 40 and the control panel 50 may be collectively defined as an input/output unit 60, in regard that they receive/transmit information from/to the user.

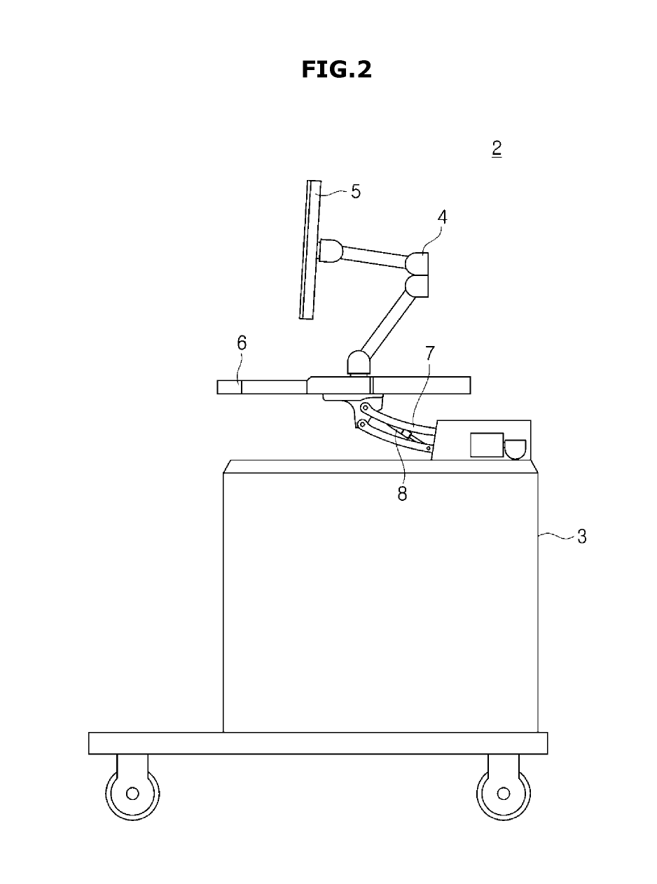

So far, the external configuration of the ultrasonic diagnostic apparatus 1 according to an embodiment of the present disclosure has been described. Hereinafter, problems of typical technology will be described with reference to FIGS. 2, 3, and 4, and then features of the present disclosure will be described,

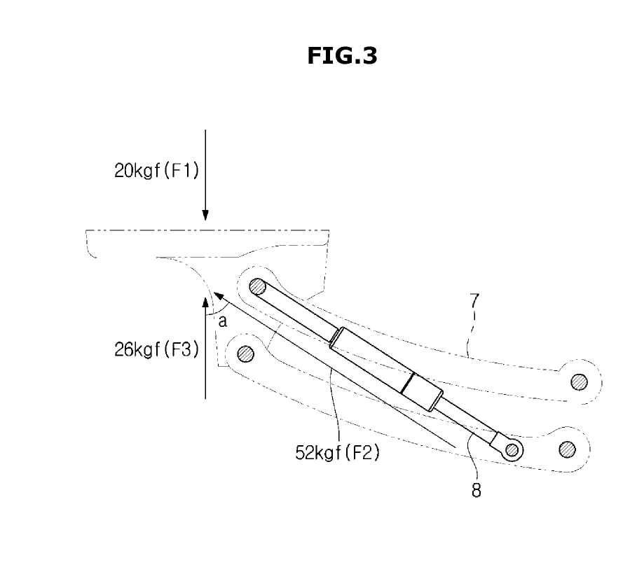

FIG. 2 is a side view of a typical ultrasonic diagnostic apparatus, and FIG. 3 is a view for describing various forces that are applied to a control panel of the typical ultrasonic diagnostic apparatus.

Referring to FIG. 2, a typical ultrasonic diagnostic apparatus 2 may include a main body 3, a display 5, and a control panel 6, and also include a connection unit 7 supporting the display 5 and the control panel 6. The connection unit 7 may include a support member 8 supporting the major part of the weight of the display 5 and the control panel 6.

Also, as shown in FIG. 3, the display 5 including the control panel 6 may apply weight to the connection unit 7 due to its own weight, and accordingly, the connection unit 7 supporting the control panel 6 and the display 7 may generate a supporting force upward.

As shown in FIG. 3, it is assumed that weight F1 generated vertically downward by the control panel 6 and the display 5 is 20 kgf, and a supporting force F2 generated in the direction of an arrow by the connection unit 7 is 52 kgf.

In this case, a force F3 applied vertically to the control panel 6 and the display 5 becomes 26 kgf, as shown in FIG. 3 (if an angle a shown in FIG. 3 is 60°, 52 kgf*cos 60=26 kgf).

Accordingly, in order for the user to move the control panel 6 and the display 5 upward, the user may need to apply a force that is greater than 6 kgf (26 kgf−20 kgf=6 kgf) to the control panel 6 or the display 5.

The force will not matter to strong men, but weak women may have difficulties in applying such a force. If the user generates an excessively great force in order to move the control panel 6 and the display 5, an unexpected accident may occur.

Also, the support member 8 supporting the control panel 6 and the display 5 may be implemented as a gas spring, instead of a normal spring, in order to simplify the configuration of the present disclosure. However, since the gas spring changes a force which it applies to the outside according to temperature, due to its characteristics, a problem may be generated according to temperature. This will be described with reference to FIG. 3, below.

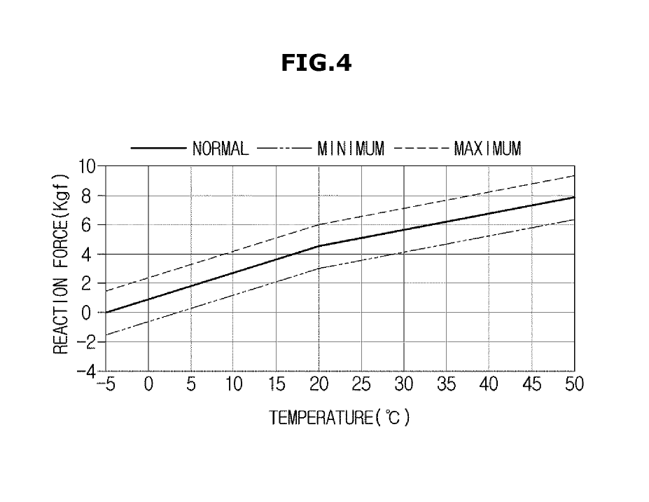

FIG. 4 is a graph showing the magnitude of a force generated by a gas spring according to a change in temperature.

The gas spring means a spring performing the function of a spring using the elasticity of gas (air or nitrogen) charged in closed space. The gas spring can change the pressure of air to maintain the stroke of spring constant regardless of the increase/decrease of weight.

The gas spring is greatly influenced by temperature since it uses gas inside a piston.

For example, if the average temperature of Korea is assumed to be 20° C., the gas spring may generate a supporting force of about 5 kgf upward in Korea, as shown in FIG. 4.

However, if an ultrasonic diagnostic apparatus manufactured in Korea moves to the equator where the outside temperature rises to about 50° C., a force generated by the gas spring may become 8 kgf due to the outside temperature, as shown in FIG. 4.

Accordingly, if an ultrasonic diagnostic apparatus manufactured to be suitable for the annual average temperature of Korea moves to hot regions, the control panel and the display may move too easily due to a great force generated by the gas spring. In this state, if a user moves the control panel upward, the main body of the ultrasonic diagnostic apparatus may move upward together with the control panel.

However, if the ultrasonic diagnostic apparatus moves to a cold region such as the South Pole or the North Pole where the outside temperature drops below zero, a force generated by the gas spring may converge toward zero. Accordingly, in this case, since a supporting force generated by the gas spring is not cancelled out by another force, the user may need to apply a great force to move the control panel and the display.

Accordingly, due to the characteristic of the gas spring, if an ultrasonic diagnostic apparatus manufactured in Korea to be suitable for the annual average temperature of Korea moves to other regions, the gas spring may generate different forces so that the user can have difficulties in moving the control panel and the display.

The present disclosure is aimed to resolve the problem, and characterized in that a user can easily move the control panel and the display in consideration of the user's physical condition and a surrounding environment. Hereinafter, the present disclosure will be described in detail with reference to the accompanying drawings.

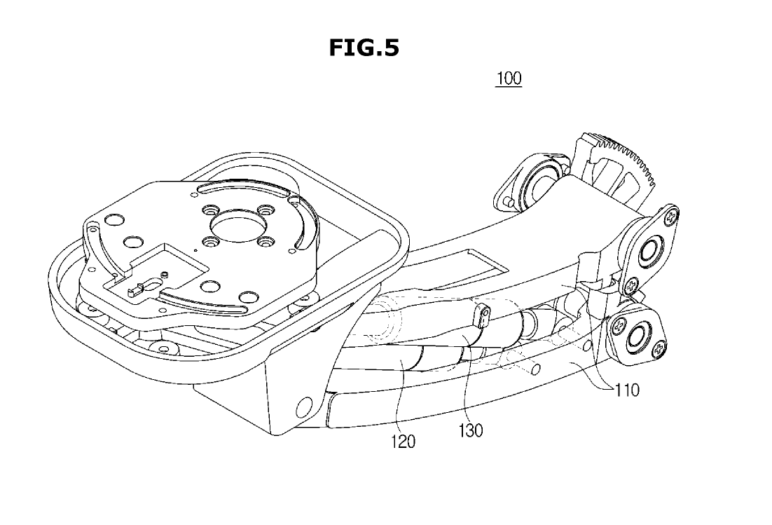

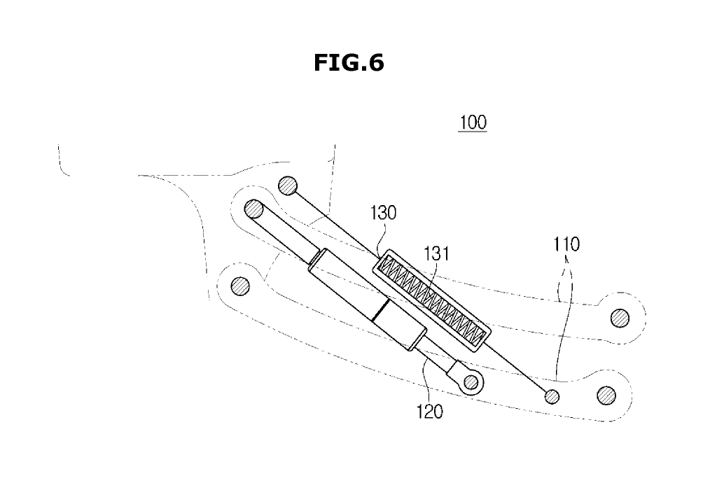

FIG. 5 is an exploded perspective view showing a part of a connection unit of an ultrasonic diagnostic apparatus according to an embodiment of the present disclosure, and FIG. 6 is an exploded perspective view showing the part of the connection unit of the ultrasonic diagnostic apparatus according to an embodiment of the present disclosure.

Referring to FIGS. 5 and 6, the connection unit 100 for adjusting the height and horizontal movement of the input/output unit 60 may include at least one arm 110 connecting the input/output unit 60 to the main body 10, a support member 120 supporting the weight of the input/output unit 60, and a connection member 130 to adjust the magnitude of a supporting force generated by the support member 120.

Also, a driving member (not shown) may be provided to move the input/output unit 60, which is not shown in the drawings. The driving member is used to move the input/output unit 60 automatically, not by the user's manual manipulation.

The support member 120 may function to support the weight of the input/output unit 60, and may function to absorb an impact that is transferred to the input/output unit 60, while functioning to fix the position of the input/output unit 60.

According to an embodiment of the present disclosure, the support member 120 may include a gas spring, although it is not limited to this. For example, the support member 120 may be any other device such as an oil damper capable of fixing the input/output unit 60.

The connection member 130 may function to adjust the magnitude of the supporting force generated by the support member 120. More specifically, the connection member 130 may generate a force in a direction that is opposite to the direction of the supporting force generated by the support member 120 so as to cancel out the magnitude of the supporting force generated by the support member 120.

Accordingly, the connection member 130 may include an elastic member. For example, the connection member 130 may include any one of a steel spring or a rubber spring, although it is not limited to these. However, the connection member 130 may be any other device capable of functioning similarly to the steel spring or the rubber spring.

Also, the connection member 130 may adjust the magnitude of the supporting force generated by the support member 120 using one or more magnetic members 132 having magnetism. Accordingly, the connection member 130 may include an object having magnetism, for example, a magnet or electromagnet.

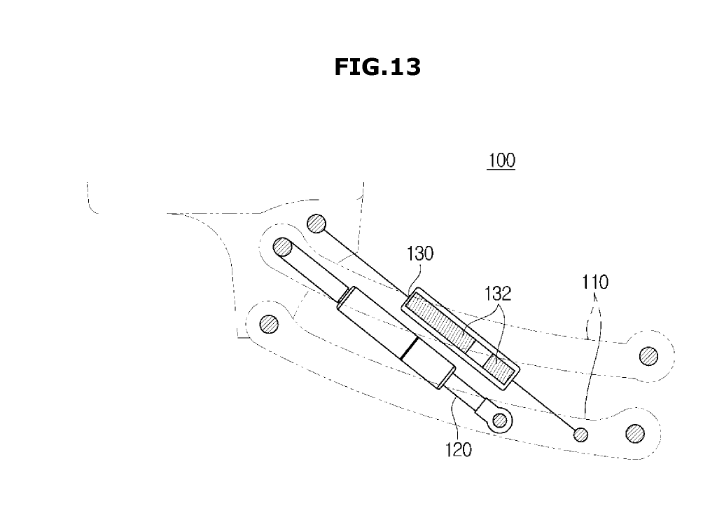

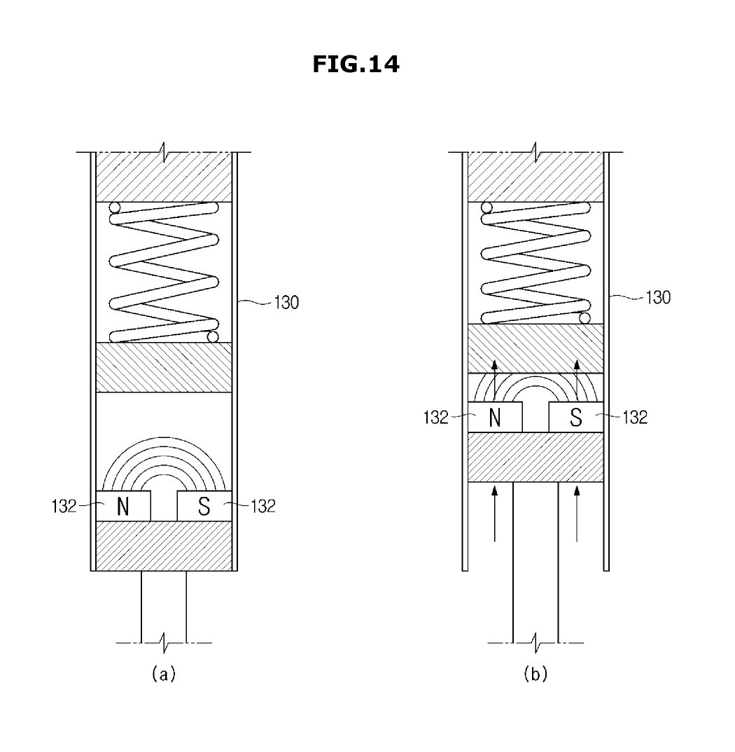

Also, the connection member 130 may wind a coil around an object having magnetism to adjust current flowing through the coil, thereby adjusting the magnitude of the supporting force generated by the support member 120. This operation will be described in detail with reference to FIGS. 13 and 14, later.

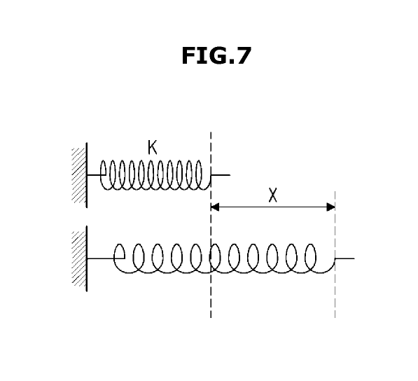

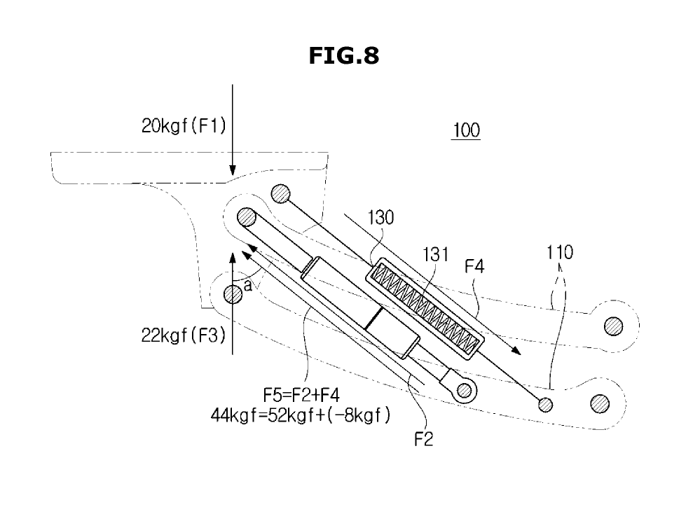

FIGS. 7 and 8 are views for describing an operation principle when an elastic member is applied to a connection member, according to an embodiment of the present disclosure, wherein FIG. 7 is a view for describing a relationship between an elastic force and an external force, and FIG. 8 shows the direction and magnitude of a force applied to the connection unit.

Referring to FIG. 7, if an external force is applied to the elastic member 131 so that the length of the elastic member 131 changes, the elastic member 131 may have a restoring force for returning to its original state due to its characteristics. If the external force increases so that the length of the elastic member 131 increases, a greater restoring force that is proportional to the length of the elastic member 131 may be generated by the elastic member 131. The force generated by the elastic member 131 may be expressed by Equation 1, below.

F=−k*x, where k is an elastic coefficient, and x is a distance. Equation (1)

Accordingly, by applying the operation principle, a user can easily adjust the magnitude of a supporting force generated by the support member 130.

For example, as shown FIG. 8, if weight F1 generated vertically downward by the input/output unit 60 is 20 kgf, a supporting force F2 generated by the support member 130 is 52 kgf, and a force F4 of −0.8 kgf is generated by the connection member 130, the magnitude of a supporting force F5 finally generated by the connection unit 120 may become 44 kgf (F5=F2+F4). Accordingly, the force F3 applied vertically to the input/output unit 60 may become 22 kgf (44*cos 60=22).

Accordingly, a force which the user needs to finally apply in order to move the input/output unit 60 upward may be 2 kgf (=22 kgf−20 kgf), which is smaller than the force of 6 kgf required in the embodiment of FIG. 3. As a result, the user can easily move the input/output unit 60.

Also, the magnitudes of the forces shown in FIG. 8 are for illustration purpose only and not for the purpose of limiting the invention. That is, it is possible to change the magnitude of a force generated by the connection member 130.

For example, it is assumed that if the magnitude of a force generated by the connection member 130 is 5 kgf when the ultrasonic diagnostic apparatus 1 is shipped, the force of 5 kgf allows a strong user to easily move the input/output unit 60.

In this case, since the user can move the input/output unit 60 too lightly, the input/output unit 60 may be moved by a distance that is longer than that intended by the user. If the user applies a strong external force to move the input/output unit 60 upward, the main body 10 may move upward.

However, since the length of the connection member 130 can be adjusted to adjust the magnitude of a supporting force generated by the support member 120, the user can adjust a force generated by the connection member 130 to 5 kgf or less to move the input/output unit 60 properly with his/her own force.

If the user is a weak woman and cannot easily move the input/output unit 60 under the condition when the ultrasonic diagnostic apparatus 1 is shipped, the user may adjust the connection member 130 so that the magnitude of a force generated by the connection member 130 becomes 5 kgf or more, thereby easily moving the input/output unit 60.

So far, the operation principle of the connection member 130 has been described. Hereinafter, a method of adjusting the length of the connection member 130 will be described with reference to the accompanying drawings.

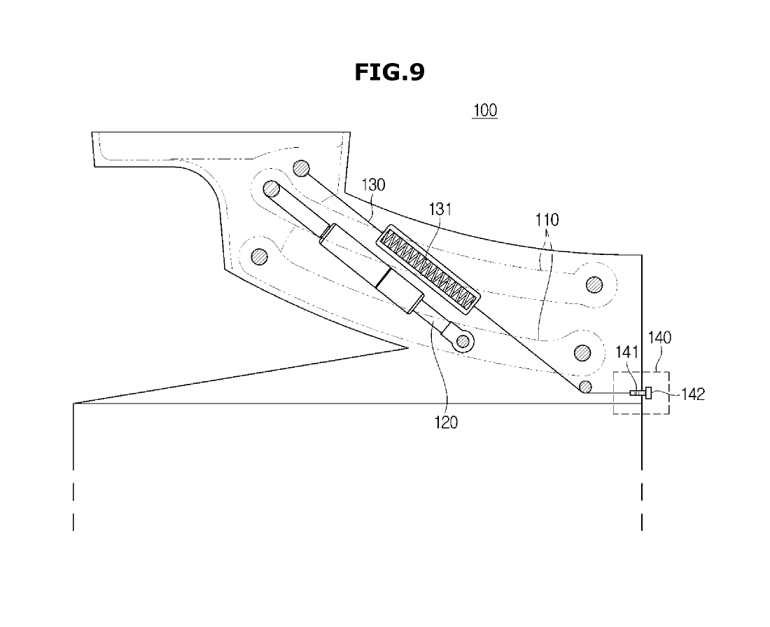

Referring to FIG. 9, a structure of the connection member 130 shown in FIG. 9 is the same as that shown in FIG. 6, except that an adjusting unit 140 for adjusting the length of the connection member 130 is further provided.

If the connection member 130 includes the elastic member 131, a user may use the adjusting unit 140 to change the length of the connection member 130, thereby adjusting the magnitude of a force generated by the connection member 130, since the magnitude of the force generated by the connection member 130 changes according to the length of the connection member 130, as described above.

For example, if a force generated by the connection member 130 is small in the condition when the ultrasonic diagnostic apparatus 1 is shipped so that the user cannot easily move the input/output unit 60, the user may use the adjusting unit 140 to lengthen the length of the connection member 130 so as to increase the magnitude of a force generated by the connection member 130.

If the magnitude of the force generated by the connection member 130 increases, the magnitude of a supporting force generated finally by the connection unit 100 may decrease so that the user can easily move the input/output unit 60.

For example, if a force generated by the connection member 130 is great in the condition when the ultrasonic diagnostic apparatus 1 is shipped so that the user can move the input/output unit 60 too lightly, the user may use the adjusting unit 140 to shorten the length of the connection member 130 so as to decrease the magnitude of a force generated by the connection member 130.

If the magnitude of the force generated by the connection member 130 decreases, the magnitude of a supporting force generated finally by the connection unit 100 may increase so that the user can move the input/output unit 60 properly with his/her own force.

Accordingly, the adjusting unit 140 may function to adjust the length of the connection member 130, as described above. The adjusting unit 140 may include an adjusting screw 141 to enable the user to easily adjust the length of the connection member 130. That is, the user may rotate the adjusting screw 141 to adjust the length of the connection member 130.

If the user rotates the adjusting screw 141 in a clockwise direction, the adjusting screw 141 may move forward so that the length of the connection member 130 is shortened to decrease the magnitude of a force generated by the connection member 130.

In contrast, if the user rotates the adjusting screw 141 in a counterclockwise direction, the adjusting screw 141 may move backward so that the length of the connection member 130 is lengthened to increase the magnitude of a force generated by the connection member 130.

Accordingly, the user may adjust the length of the connection member 130 by rotating the adjusting screw 141.

Also, a fixing unit (not shown) may be provided to prevent the adjusting unit 140 from rotating in the reverse direction after the length of the connection member 130 is changed by the adjusting unit 140.

The fixing unit may include a fixing screw for preventing reverse rotation or a sawtoothed head for preventing reverse rotation, although it is not limited to these. That is, the fixing unit may include any other device capable of functioning similarly to the fixing screw or the sawtoothed head.

Also, the adjusting unit 140 may further include a handle 142 coupled with the adjusting screw 141 to enable the user to easily manipulate the adjusting screw 141. Since the user has difficulties in rotating the adjusting unit 140 with only the adjusting screw 141, the handle 142 may be coupled with the adjusting screw 141 so that the user can easily adjust the adjusting screw 141.

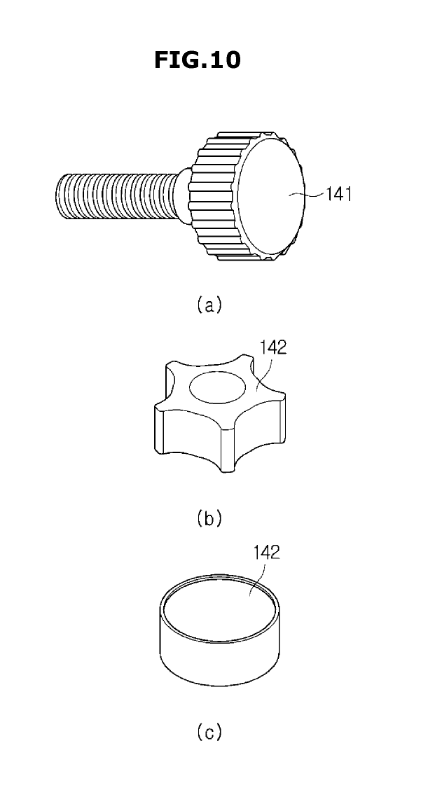

The adjusting screw 141 may be a screw as shown in FIG. 10A, and the handle 142 may be configured to include any one of a knob and a jog-dial as shown in FIGS. 10B and 10C, although not limited to these. That is, the handle 142 may be any handle capable of functioning similarly to the knob or the jog-dial.

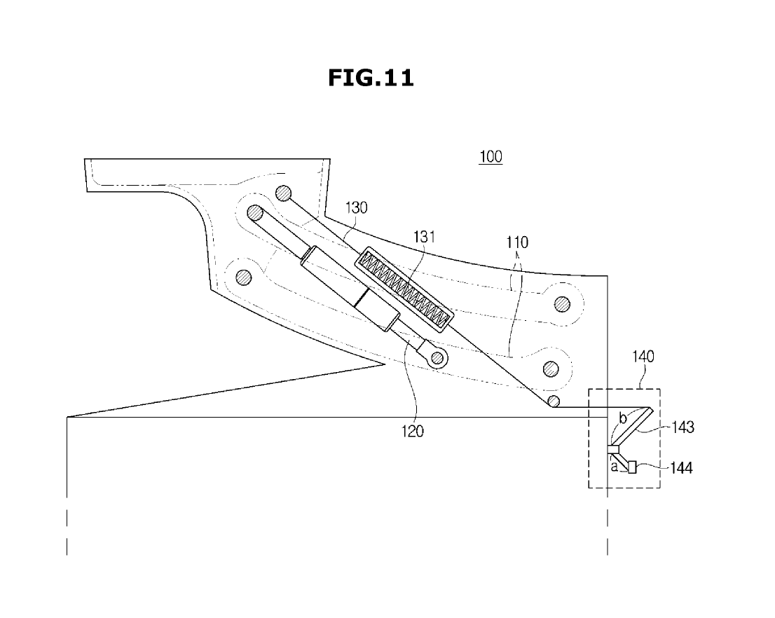

FIG. 11 shows a connection member including a lever handle, according to another embodiment of the present disclosure, wherein the length of the connection member can be adjusted based on the principle of the lever.

Referring to FIG. 11, one end of the lever handle 143 may be connected to the connection member 130, and the other end of the lever handle 143 may include a handle 144 to enable a user to manipulate the lever handle 143.

The user can easily adjust the length of the connection member 130 based on the principle of the lever.

Referring to FIG. 8, since the length of a portion a is shorter than that of another portion b, the length of the portion a can be easily adjusted by adjusting the length of the portion b. That is, if the user moves the portion b by 5 cm, the portion a can be moved by 10 cm according to the principle of the lever so that the user can easily adjust the length of the connection member 130 using the lever handle 143.

Also, in order for the user to more easily adjust the length of the connection member 130, a screw 144 may be additionally connected to one end of the lever handle 143.

That is, the user may manipulate the lever handle 143 by rotating the screw 144, and if the lever handle 143 moves, the length of the connection member 130 can be adjusted, as described above.

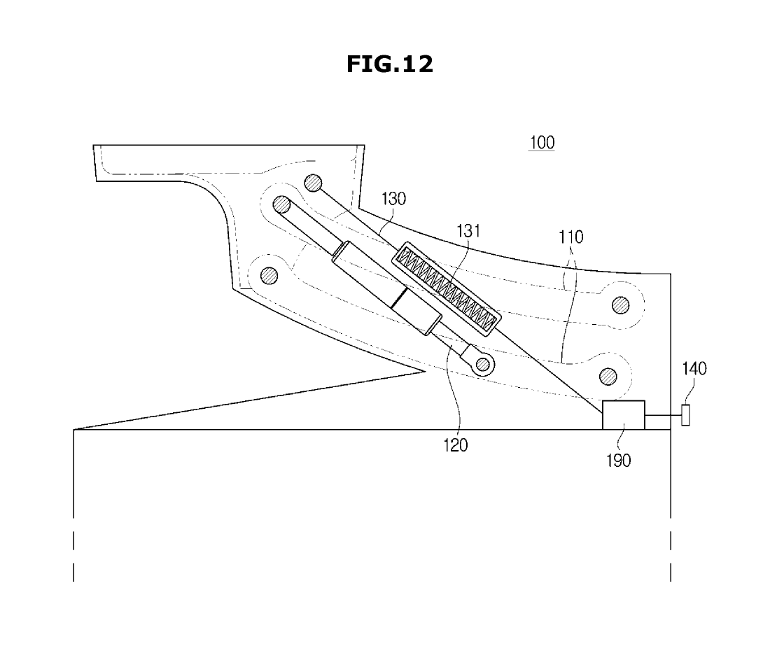

FIG. 12 is a view for describing a case of adjusting the length of a connection member using a motor, according to another embodiment of the present disclosure.

Although a user can adjust the length of the connection member 130, a motor 190 may be used to easily adjust the length of the connection member 130 through a simple manipulation.

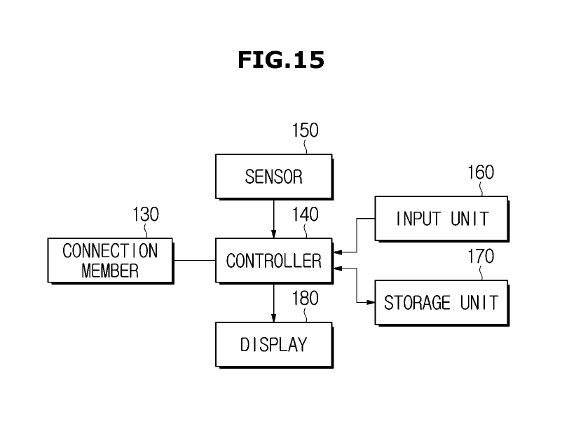

Although not shown in FIG. 12, the user may input information about the length of the connection member 130 using an input unit 160 (see FIG. 15) included in the adjusting unit 140, and the controller 140 may control the motor 190 according to the input information to adjust the length of the connection member 130.

Also, the controller 140 may automatically adjust the motor 190 based on temperature measured by a sensor 150 (see FIG. 15) to adjust the length of the connection member 130.

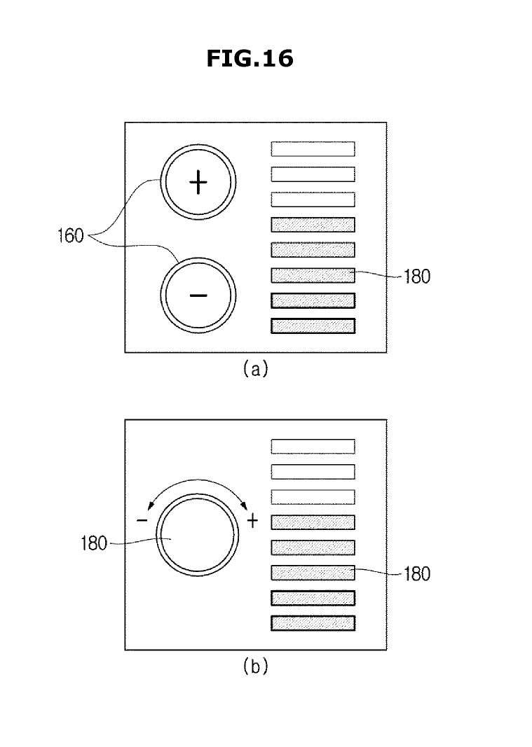

Accordingly, although the user does not adjust the length of the connection member 130 according to his/her own force before using the ultrasonic diagnostic apparatus 1, the controller 140 can set a condition in which the user can easily move the input/output unit 60. The controller 140 and the sensor 150 will be described in detail with reference to FIG. 16, later.

So far, the connection member 130 including the elastic member 131 has been described. Hereinafter, the operation principle of the connection member 130 when the connection member 130 includes the magnetic members 132, instead of the elastic member 131, will be described.

FIG. 13 shows a connection member including one or more magnetic members, according to an embodiment of the present disclosure, and FIG. 14 is a view for describing a force generated by the connection member including the magnetic members.

Referring to FIG. 13, the connection unit 100 according to an embodiment of the present disclosure may include the at least one arm 110 connecting the input/output unit 60 to the main body 10, the support member 120 to support the weight of the input/output unit 60, and the connection member 130 to adjust the magnitude of a supporting force generated by the support member 120, wherein the connection member 130 may include the magnetic members 132 having magnetism.

The individual components of the connection unit 100 have been described above with reference to FIGS. 5 and 6, and in the embodiment of FIG. 13, the magnitude of a supporting force generated by the support member 120 may be adjusted by the magnetic members 132, instead of the elastic member 131.

Accordingly, the magnetic members 132 may include an object having magnetism, for example, a magnet or electromagnet. However, the magnetic members 132 may include any other objects functioning similarly to the magnet or the electromagnet.

FIG. 14 is a view for describing a method of generating a magnetic force using the magnetic members shown in FIG. 13.

Since the magnetic field lines are directed from the N pole to the S pole, the magnetic field lines may be made as shown in FIG. 14A, as long as there is no interference.

However, if the distance between magnetic members is short as shown in FIG. 14B, the magnetic field lines may lose their original shapes, and in this case, the magnetic field lines may generate a force for restoring their original shapes, like the above-described elastic force. Accordingly, if the distance between the magnetic members is short as shown in FIG. 14B, a force may be generated upward, and as the interval between the magnetic field lines is narrower, the stronger force may be generated.

Accordingly, the connection member 130 can adjust the magnitude of a supporting force using the principle.

If a user wants to increase the magnitude of a force generated by the magnetic members 132, the user may push the magnetic members 132 upward to decrease the magnitude of a total supporting force. In contrast, if the user wants to decrease the magnitude of a force generated by the magnetic members 132, the user may pull the magnetic members 132 downward to increase the magnitude of a total supporting force.

In FIG. 14, a case of adjusting the interval by moving the magnetic members 132 is shown, however, any other means capable of generating a magnetic force by adjusting the interval between the magnetic members 132 or moving the magnetic members 132 may be used.

Also, although not shown in the drawings, the magnetic members 132 may further include a coil. If the coil is wound around the magnetic members 132 and then current flows through the coil, a strong magnetic field may be formed to generate a magnetic force.

If current flowing through the coil increases, a strong magnetic force may be generated, and if current flowing through the coil deceases, a weak magnetic force may be generated. The current flowing through the coil may be adjusted by the user through the input unit 160 which will be described later.

FIG. 15 is a block diagram showing components constituting a part of an ultrasonic diagnostic apparatus according to another embodiment of the present disclosure.

Referring to FIG. 15, an ultrasonic diagnostic apparatus 1 may include the sensor 150 configured to adjust the length of the connection member 130 described above, the input unit 160 configured to receive information about the connection member 130 from a user, a storage unit 170 configured to store the information about the connection member 130, a display 180 configured to display the information about the connection member 130, and a controller 140 configured to control the input unit 160, the storage unit 170, the display 180, and the connection member 130.

The sensor 150, which is a component for measuring the inside/outside temperature of the ultrasonic diagnostic apparatus 1, may function to transmit the measured temperature to the controller 140. Accordingly, the sensor 150 may include a thermometer for measuring temperature, and also include various devices required for measuring the temperature.

Also, the controller 140 may adjust the length of the connection member 130 automatically based on the temperature measured by the sensor 150. That is, if the temperature measured by the sensor 150 is high so that the magnitude of a supporting force generated by the gas spring is great, the controller 140 may adjust the length of the connection member 130 automatically to decrease the magnitude of the force generated by the gas spring.

In contrast, if the temperature measured by the sensor 150 is low so that the magnitude of a supporting force generated by the gas spring is small, the controller 140 may adjust the length of the connection member 130 automatically to increase the magnitude of the force generated by the gas spring so that the user can move the input/output unit 60 properly according to his/her condition.

Also, the controller 140 may adjust the length of the connection member 130 automatically according to a predetermined condition. That is, if the user has set the length of the connection member 130 in a predetermined temperature range in advance, the controller 140 may detect temperature measured by the sensor 150 in real time to adjust the length of the connection member 130.

In this case, since the user does not need to adjust the length of the connection member 130 in consideration of his/her physical condition or a surrounding environment, the user can move the input/output unit 60 conveniently.

The input unit 160 may function to receive information about the connection member 130 from the user, and the display 180 may display the information about the connection member 130, that is, the magnitude of a force generated by the connection member 130.

More specifically, if the connection member 130 uses the elastic member 131, the user may input a command for setting the length of the connection member 130, and if the connection member 130 uses the magnetic members 132, the user may input a command for setting the interval between the magnetic members 132 or a command for setting the magnitude of current flowing through the coil.

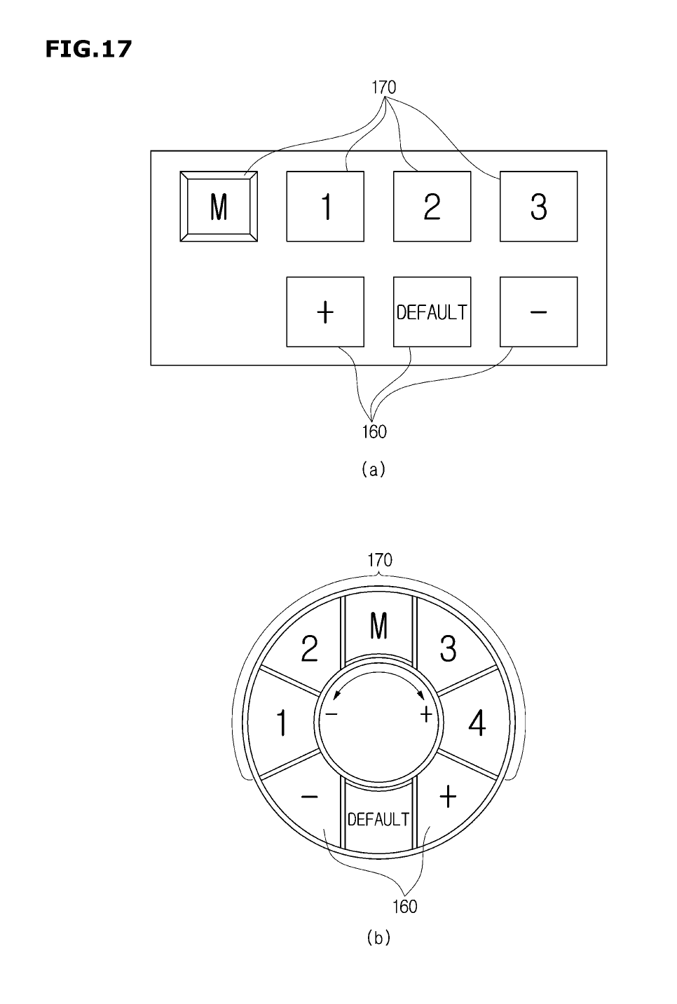

Also, the user may manipulate at least any one of a button, a switch, a knob, and a jog-dial, instead of inputting a value, as shown in FIG. 17.

For example, the user may adjust the magnitude of a force generated by the connection member 130 using a (+) button and a (−) button, as shown in FIG. 16A, or may adjust the magnitude of the force using a jog-dial, as shown in FIG. 16B.

More specifically, if the user wants to increase the magnitude of a force generated by the connection member 130, the user may press the (+) button or rotate the jog-dial to the right, and if the user wants to decrease the magnitude of a force generated by the connection member 130, the user may press the (−) button or rotate the jog-dial to the left, thereby adjusting the magnitude of a force generated by the connection member 130.

The display 180 may inform the user of the magnitude of a force generated by the connection member 130 using a display device exposed to the outside of the ultrasonic diagnostic apparatus 1, as shown in FIGS. 16A and 16B. As shown in FIG. 16, the magnitude of the force may be represented by lighting LEDs, however, the magnitude of the force may be represented by a figure. That is, a method of representing the magnitude of the force is not limited.

Accordingly, the display 180 may include various kinds of LEDs or lasers, and various kinds of Printed Circuit Boards (PCBs).

The storage unit 170 may enable the user to store the information about the connection member 130. In many cases, the ultrasonic diagnostic apparatus 1 is used by many peoples, and accordingly, a current user may be greatly influenced by environment settings done by the previous user.

Accordingly, if each user stores the length of the connection member 130 most suitable for him/her, and loads the stored length of the connection member 130 as necessary, the user will not need to perform environment settings whenever using the ultrasonic diagnostic apparatus 1.

As shown in FIGS. 17A and 17B, the user may set an environment most suitable for him/her using the buttons of the input unit 160, and then store the set environment in the storage unit 170 using a M button. Accordingly, the user can load the environment suitable for him/her simply by pressing a figure button, although another user changed the length of the connection member 130 when using the ultrasonic diagnostic apparatus 1.

Accordingly, the storage unit 170 may include various kinds of electronic devices and circuits, such as a hard disk or Random Access Memory (RAM), in order to store information.

FIG. 17 shows a basic structure due to the limitation of drawings, however, more buttons may be provided, and also various modifications are possible.

So far, the features and effects of the present disclosure have been described based on various embodiments of the present disclosure.

Since the typical ultrasonic diagnostic apparatus does not consider a user's physical condition and a surrounding environment, the user may have difficulties in moving the control panel and the display of the ultrasonic diagnostic apparatus.

However, according to the present disclosure, since a user can freely adjust the magnitude of a supporting force generated against the control panel and the display in consideration of his/her physical condition and a surrounding environment, the user can easily move the control panel and the display.

In the ultrasonic diagnostic apparatus according to the present disclosure, since the magnitude of a supporting force generated by the support member supporting the input/output unit can be adjusted in consideration of a user's physical condition or a surrounding environment, the user can easily move the input/output unit.

As described above, although the present disclosure have been described with reference to the limited embodiments and the drawings, those skilled in the art will appreciate that various modifications and changes are possible from the above description. For example, even if the above-described technologies are performed in a sequence differing from that in the description and/or even if the components of the above-described system, structure, apparatus, and circuit are coupled or combined in a way differing from that of the above-described method, or even if the components are replaced or substituted with other components or equivalents, suitable results may be achieved. Therefore, it should be understood that the other embodiments and examples, and equivalents of the accompanying claims are included in the scope of the accompanying claims.

1. An ultrasonic diagnostic apparatus comprising:
a main body;
at least one input/output unit coupled with the main body, and configured to receive information from a user or to output information received from the main body; and
a connection unit configured to connect the main body to the input/output unit,
wherein the connection unit comprises a support member configured to support the input/output unit, and at least one connection member configured to adjust a magnitude of a supporting force generated by the support member.

2. The ultrasonic diagnostic apparatus according to claim 1, wherein the connection member generates a force in a direction in which the supporting force generated by the support member is cancelled out to thus adjust the magnitude of the supporting force.

3. The ultrasonic diagnostic apparatus according to claim 1, wherein the support member comprises a gas spring.

4. The ultrasonic diagnostic apparatus according to claim 1, wherein the connection member comprises an elastic member having elasticity.

5. The ultrasonic diagnostic apparatus according to claim 1, further comprising an adjusting unit coupled with one end of the connection member, and configured to adjust a length of the connection member.

6. The ultrasonic diagnostic apparatus according to claim 5, wherein the adjusting unit comprises an adjusting screw configured to rotate to adjust the length of the connection member.

7. The ultrasonic diagnostic apparatus according to claim 6, wherein the adjusting unit further comprises a handle coupled with the adjusting screw, and rotation of the adjusting screw is adjusted by the handle.

8. The ultrasonic diagnostic apparatus according to claim 5, wherein the adjusting unit comprises a lever handle configured to adjust the length of the connection member based on the principle of the lever.

9. The ultrasonic diagnostic apparatus according to claim 5, further comprising a fixing unit configured to fix the adjusting unit or to prevent reverse rotation of the adjusting unit.

10. The ultrasonic diagnostic apparatus according to claim 1, wherein the connection member comprises a single or a plurality of magnetic members having magnetism.

11. The ultrasonic diagnostic apparatus according to claim 10, wherein in the connection member, the magnitude of the supporting force is adjusted according to an interval between the plurality of magnetic members.

12. The ultrasonic diagnostic apparatus according to claim 10, wherein the connection member further comprises a coil wound around the single or the plurality of magnetic members, and the magnitude of the supporting force is adjusted according to a magnitude of current flowing through the coil.

13. The ultrasonic diagnostic apparatus according to claim 12, further comprising an input unit configured to receive information about an interval between the plurality of magnetic members or information about a magnitude of the current flowing through the coil, from the user.

14. The ultrasonic diagnostic apparatus according to claim 13, further comprising a controller configured to control the interval between the plurality of magnetic member or the magnitude of the current flowing through the coil, according to the received information.

15. The ultrasonic diagnostic apparatus according to claim 4, further comprising an input unit configured to receive information about the length of the connection member from the user.

16. The ultrasonic diagnostic apparatus according to claim 13, wherein the input unit comprises one of a button, a switch, a knob, and a jog-dial.

17. The ultrasonic diagnostic apparatus according to claim 15, further comprising a motor configured to adjust the length of the connection member, and a controller configured to control operation of the motor,
wherein the controller adjusts the length of the connection member using the motor based on the received information.

18. The ultrasonic diagnostic apparatus according to claim 1, further comprising a sensor configured to measure outside temperature, and a controller configured to adjust the length of the connection member based on the outside temperature measured by the sensor.

19. The ultrasonic diagnostic apparatus according to claim 1, further comprising a storage unit configured to store information about the connection member set by the user, and a display configured to display a magnitude of a force generated by the connection member.

20. The ultrasonic diagnostic apparatus according to claim 1, wherein the input/output unit comprises one of a control panel and a display.
